(19) United States
(12) Patent Application Publication
(10) Pub. No.: US 2022/0265995 A1
(43) Pub. Date: Aug. 25, 2022

(54) Electrical Stimulation Device and Method

(71) Applicant: Nippon Telegraph and Telephone Corporation, Tokyo (JP)

(72) Inventors: Toshishige Shimamura, Tokyo (JP); Rena Nakatsuji, Tokyo (JP); Hitoshi Okikawa, Tokyo (JP); Yuzo Ishii, Tokyo (JP); Yoshiyuki Doi, Tokyo (JP); Nobutomo Yoshihashi, Tokyo (JP)

(21) Appl. No.: 17/628,747

(22) PCT Filed: Jul. 23, 2019
(86) PCT No.: PCT/JP2019/028798

§ 371 Date: Jan. 20, 2022

(51) Int. Cl. A61N 1/04

(52) U.S. Cl. CPC A61N 1/0484

(57) ABSTRACT

An embodiment electrical stimulation device includes a designation circuit configured to designate an electrode at which myoelectric potential is measured from the plurality of electrodes for measuring myoelectric potential in the state in which the plurality of electrodes are fitted on the surface of the body of a target person. The electrical stimulation device also includes an application circuit configured to apply an electrical stimulus to the electrode designated by the designation circuit. The designation circuit designates, for example, an electrode at which a myoelectric potential higher than a preset reference value is measured, from the plurality of electrodes. The electrical stimulation device may also include a storage circuit configured to store identification information for identifying an electrode targeted for application. The application circuit applies an electrical stimulus to an electrode designated by the designation circuit from electrodes identified in accordance with identification information stored in the storage circuit.

### CROSS-REFERENCE TO RELATED APPLICATION

This application is a national phase entry of PCT Application No. PCT/JP2019/028798, filed on Jul. 23, 2019, which application is hereby incorporated herein by reference.

### TECHNICAL FIELD

The present invention relates to an electrical stimulation device and an electrical stimulation method.

### BACKGROUND

Technologies of designating a body part with a need for rehabilitation by comparing electrical signals of the right and left sides of the body are known. Other technologies for assisting rehabilitation by applying electrical stimuli as support are also known. As such, in the rehabilitation field, electrical stimulation therapy is utilized to, for example, relieve pain or to improve muscular strength (see, e.g., Non-Patent Literature 1, 2, 3, and 4).

### CITATION LIST

### Non-Patent Literature

Non-Patent Literature 1: G-TES (features of G-TES), Introduction of G-TES using B-SES (muscle electrical stimulation), Homer Ion Co., Ltd., 2019, [searched on Jul. 5, 2019], http://www.homerion.co.jp/products/g-tes.html).

Non-Patent Literature 2: IVES plus GD-611/IVES GD-612, Electrical Stimulator GD-611 IVES/IVES, OG Wellness Technologies Co., Ltd., 2019, [searched on Jul. 5, 2019], (https://www.og-wellness.jp/product/medical/gd611-612).

Non-Patent Literature 3: Biomonitor ME6000 (8 CH), Nihon Medix Co., Ltd., 2019, [searched on Jul. 5 , 2019], (https://www.nihonmedix.co.jp/products/details/prd_000042.php).

Non-Patent Literature 4: “Uses of muscular strength/function evaluation measurement devices”, Nihon Medix Co., Ltd., 2019, [searched on Jul. 5 , 2019], (https://www.nihonmedix.co.jp/support/04assessment_index.html).

### SUMMARY

### Technical Problem

However, when electrical stimulation therapy is performed, specialists' knowledge is necessary to designate where to apply stimuli and the treatment cannot be carried out without instructions by specialists. The effect of treatment varies among specialists in accordance with their technical levels, because the specialists designate pails to be stimulated by using rules based on their experiences. Moreover, private practitioners at clinics tend to lack knowledge about rehabilitation, and thus, switching from dedicated rehabilitation facilities to home rehabilitation care cannot always be carried out in a smooth manner.

The embodiments of the present invention have been made to address the problems, and an object thereof is to relatively easily apply electrical stimuli to a pail with a need for rehabilitation.

### Means for Solving the Problem

An electrical stimulation device according to an embodiment of the present invention includes a plurality of electrodes for measuring myoelectric potential in the state in which the plurality of electrodes are fitted on a body surface of a target person, a designation circuit configured to designate, from the plurality of electrodes, an electrode at which myoelectric potential is measured, and an application circuit configured to apply a signal to the electrode designated by the designation circuit.

An electrical stimulation method according to an embodiment of the present invention includes a designation step of designating an electrode at which myoelectric potential is measured from a plurality of electrodes for measuring myoelectric potential in the state in which the plurality of electrodes are fitted on a body surface of a target person and an application step of applying a signal to the electrode designated in the designation step.

### Effects of Embodiments of the Invention

With the configuration described above, embodiments of the present invention can relatively easily apply electrical stimuli to a part with a need for rehabilitation.

### BRIEF DESCRIPTION OF THE DRAWINGS

FIG. 1 is a configuration diagram illustrating a configuration of an electrical stimulation device according to an embodiment of the present invention.

FIG. 2 is a configuration diagram illustrating a configuration of a garment including a plurality of electrodes.

FIG. 3A is a perspective view illustrating a configuration of a bandage including the plurality of electrodes.

FIG. 3B is a side view illustrating a configuration of a part of the bandage including the plurality of electrodes.

FIG. 4A is a flowchart illustrating an electrical stimulation method according to the embodiment of the present invention.

FIG. 4B is a flowchart illustrating another electrical stimulation method according to the embodiment of the present invention.

FIG. 4C is a flowchart illustrating still another electrical stimulation method according to the embodiment of the present invention.

FIG. 5 is a configuration diagram illustrating a configuration of a control device of a first practical example.

FIG. 6 is a flowchart illustrating a basic operation of the control device of the first practical example.

FIG. 7 is a configuration diagram illustrating a configuration of a control device of a third practical example.

FIG. 8 is a flowchart illustrating an operation of a determination unit of the control device according to the third practical example.

FIG. 9 is a flowchart illustrating a determination method according to a fourth practical example.

FIG. 10 is a configuration diagram illustrating a configuration of a control device of a fifth practical example.

FIG. 11 is a configuration diagram illustrating a configuration of the server according to a seventh practical example of the present invention.

FIG. 12 is a configuration diagram illustrating another data configuration of the server according to the seventh practical example of the present invention.

FIG. 13 is a configuration diagram illustrating still another data configuration of the server according to the seventh practical example of the present invention.

FIG. 14 is a configuration diagram illustrating a hardware configuration of an electrical stimulation application device according to the embodiment of the present invention.

### DETAILED DESCRIPTION OF ILLUSTRATIVE EMBODIMENTS

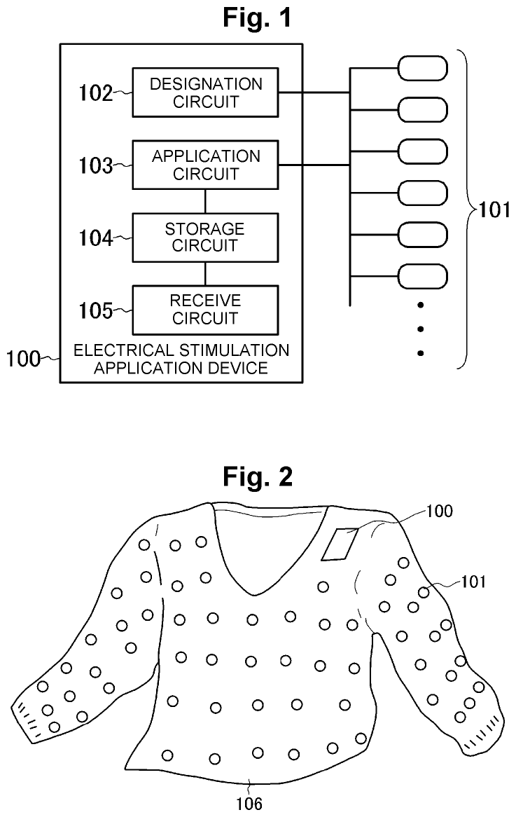

Hereinafter, an electrical stimulation device according to an embodiment of the present invention will be described with reference to FIG. 1. The electrical stimulation device includes an electrical stimulation application device too and a plurality of electrodes tot for measuring myoelectric potential in the state in which the plurality of electrodes tot are fitted on the surface of the body of a target person. The electrical stimulation application device too includes a designation circuit 102 configured to designate from the plurality of electrodes tot an electrode at which myoelectric potential is measured and an application circuit 103 configured to apply a signal (electrical stimulus) to the electrode designated by the designation circuit 102. The designation circuit 102 designates, for example, an electrode at which a myoelectric potential higher than a preset reference value is measured, from the plurality of electrodes tot.

The designation circuit 102 may include, for example, a measurement function, an identification function, and a determination function. The measurement function is a function of measuring respective electric potentials generated at each of the plurality of electrodes 101. The identification function is a function of identifying each of the plurality of electrodes 101. The determination function is a function of determining, with respect to each electrode identified by the identification function, whether the electric potential measured at the electrode by the measurement function exceeds a particular value that is usually considered as a myoelectric potential.

The electrical stimulation application device too may also include a storage circuit 104 configured to store identification information for identifying an electrode targeted for application. In this case, the application circuit 103 applies an electrical stimulus to an electrode designated by the designation circuit 102 from electrodes identified in accordance with identification information stored in the storage circuit 104. The electrical stimulation application device too may also include a receive circuit 105. The receive circuit 105 receives an instruction about a target electrode of the plurality of electrode tot and stores identification information identifying the electrode of the received instruction in the storage circuit 104.

The plurality of electrodes tot may be provided at, for example, a cloth and can be fitted on the surface of the body of a target person by covering the surface of the body of the target person with the cloth. The cloth is stretchable and easy to fit any part including joints of human bodies. The cloth may be, for example, a garment 106 such as a shirt as illustrated in FIG. 2. The garment 106 can be used with the plurality of electrodes tot fixed to the garment 106, and also together with the electrical stimulation application device too attached to the garment 106. FIG. 2 does not illustrate wiring lines connecting the electrical stimulation application device too and the plurality of electrodes tot of the garment 106. The cloth may especially cover an arm or leg joint. The cloth may be formed as a glove.

The plurality of electrodes 101 are not previously fitted at portions to be fed with electrical stimuli on the surface of the body of a target person. After the plurality of electrodes tot are fitted on the surface of the body of a target person, an electrode in contact with the designation circuit to be fed with an electrical stimulus is designated (chosen) on the target person (human body), such that a part to be fed with an electrical stimulus on a human body can be easily selected in a flexible manner. Hence, it is preferable that the plurality of electrodes tot be positioned at a cloth for covering a human body such as the garment 106 at constant density.

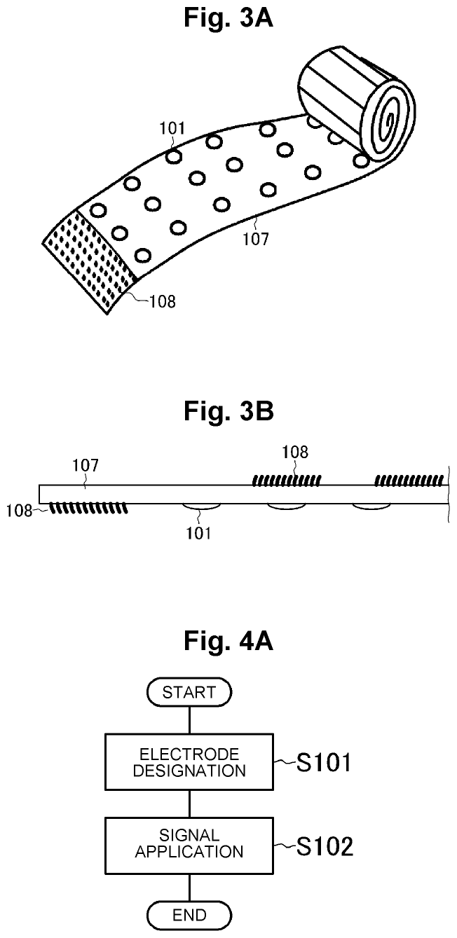

The cloth may be formed as a bandage 107 as illustrated in FIGS. 3A and 3B. The bandage 107 includes an attachment portion 108 such as a hook-and-loop fastener, so that the bandage 107 can be fitted to a part of a human body in an attachable and detachable manner. The attachment portion 108 may be formed by using a snap button. It is preferable that the attachment portion 108 be formed by a material that is deformable and attachable at any position on the attaching surface, such as a hook-and-loop fastener. The bandage 107 has advantages in which a prepared cloth of one shape can be used for both measurement of any part of a human body and application of electricity, the prepared cloth of one shape can be stocked in common for all parts, and manufacturing costs can be reduced. A plurality of attachment portions 108 may be provided at given intervals at the bandage 107. As such, the attachment portions 108 can be arranged throughout the large area of the bandage 107, while the bandage 107 can still be freely stretched. Cloths in these shapes can be more effectively used with the underlying method for designating measurement and application parts described later.

In the electrical stimulation device according to the present embodiment, the designation circuit 102 designates an electrode at which myoelectric potential is measured, and thus, it is possible to relatively easily apply electrical stimuli to a part with a need for rehabilitation.

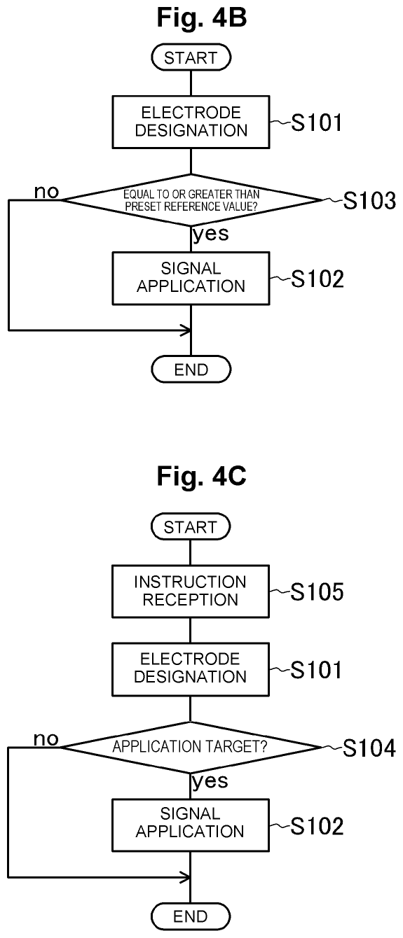

Next, an electrical stimulation method according to the embodiment of the present invention will be described by using a flowchart in FIG. 4A. Firstly, in step Sioi of the electrical stimulation method, the designation circuit 102 designates an electrode at which myoelectric potential is measured from the plurality of electrodes 101 for measuring myoelectric potential in the state in which the plurality of electrodes 101 are fitted on the surface of the body of a target person (designation step). Next, in step S102, the application circuit 103 applies an electrical stimulus to the designated electrode (application step).

Next, another electrical stimulation method according to the embodiment of the present invention will be described by using a flowchart in FIG. 4B. Firstly, in step S101, the designation circuit 102 designates an electrode at which a myoelectric potential is measured from the plurality of electrodes 101 Next, in step S103, the designation circuit 102 determines whether the myoelectric potential measured at the designated electrode is higher than a preset reference value from the plurality of electrodes 101. When the measured myoelectric potential is equal to or higher than the preset reference value (yes in step S103), the application circuit 103 applies an electrical stimulus to the designated electrode in step S102. By contrast, when the measured myoelectric potential is lower than the preset reference value (no in step S103), the process ends without applying any electrical stimulus.

Next, still another electrical stimulation method according to the embodiment of the present invention will be described by using a flowchart in FIG. 4C. Firstly, in step S105, an instruction about a target electrode of the plurality of electrode 101 is received, and identification information identifying the electrode of the received instruction is stored in the storage circuit 104 (reception step). Next, in step S101, the designation circuit 102 designates an electrode at which myoelectric potential is measured from the plurality of electrodes 101.

Next, in step S104, the designation circuit 102 determines whether the designated electrode is the electrode identified by the identification information stored in the storage circuit 104. When the designated electrode is the electrode identified by the identification information stored in the storage circuit 104 (yes in step S104), the application circuit 103 applies an electrical stimulus to the designated electrode in step S102. By contrast, when the designated electrode is not the electrode identified by the identification information stored in the storage circuit 104 (no in step S104), the process ends without applying any electrical stimulus.

Hereinafter, more details will be described by using practical examples.

### First Practical Example

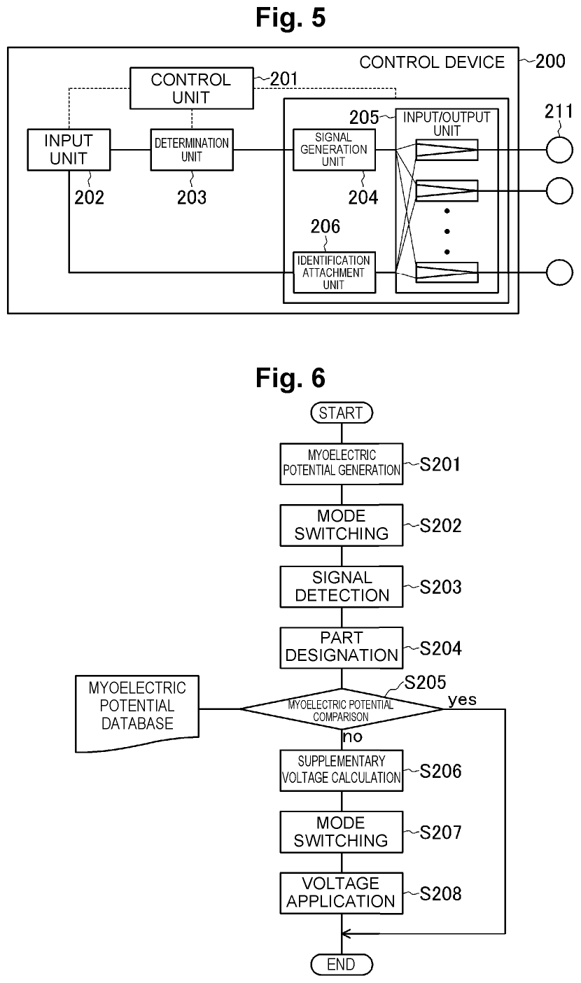

Firstly, a first practical example will be described with reference to FIG. 5. The first practical example describes a control device 200 that operates as the electrical stimulation application device. The control device 200 processes and records myoelectric potentials measured at the plurality of electrodes 211. The control device 200 also designates (selects) an electrode to be fed with electricity from the plurality of electrodes 211 and applies electricity to the electrode. The control device 200 is coupled to each of the plurality of electrodes 211 by wiring lines. The control device 200 includes an input unit (e.g., input circuit) 202, a determination unit (e.g., determination circuit) 203, a signal generation unit (e.g., signal generation circuit) 204, an input/output unit (e.g., input/output circuit) 205, and an identification attachment unit (e.g., identification assignment circuit) 206. The input unit 202, the determination unit 203, the input/output unit 205, and the identification attachment unit 206 form a designation circuit. The determination unit 203, the signal generation unit 204, and the input/output unit 205 form an application circuit. For ease of description, the following is a description of processing for one electrode, but in practical operation myoelectric potential is generated at a plurality of electrodes and voltage is applied to the plurality of electrodes at the same time, which means that the processing is performed in parallel for a plurality of electrodes.

The input unit 202 receives information about a myoelectric signal generated by a human body together with identification information of an electrode. A control unit 201 controls the functional blocks of the input unit 202, the determination unit 203, the signal generation unit 204, the input/output unit 205, and the identification attachment unit 206 to operate. Although the control unit 201 controls the functional blocks to operate, the following description is made in accordance with operations of the functional blocks without a description of the control operation.

The determination unit 203 has a function of determining whether to apply electricity to the electrode 211 or how much voltage needs to be applied. The signal generation unit 204 has a function of selecting the electrode corresponding to identification (ID) information that the determination unit 203 determines to be fed with electricity, receiving electricity (voltage and current) supplied by an external power supply not illustrated in the drawing, and applying the electricity to the electrode in accordance with an instruction provided by the determination unit 203.

The input/output unit 205 divides electricity (e.g., a signal) outputted to an electrode and electricity (e.g., myoelectric potential) inputted from an electrode to the control device 200. When electricity is inputted from an electrode, the input/output unit 205 plays two kinds of roles. The first role is transferring wiring line information associated with the electrode to the identification attachment unit 206. The second role is supplying current and voltage from the signal generation unit 204 to a designated electrode through a wiring line connected to the electrode. The input/output unit 205 includes a switch for changing between a detection mode for measuring myoelectric potential and an application mode for applying voltage to reduce a deficit. The control unit 201 controls the input/output unit 205 to switch between these modes.

The identification attachment unit 206 has a function of storing IDs (e.g., electrode IDs) of electrodes in association with wiring line information, receiving wiring line information and myoelectric potential data from the input/output unit 205, attaching a corresponding electrode ID to the wiring line information and myoelectric potential data, and transferring to the input unit 202 the wiring line information and myoelectric potential data in association with the electrode ID.

A basic operation of this practical example will be described with reference to FIG. 6. In this practical example, for example, a subject wears the garment 106 described with reference to FIG. 2 or ties the bandage 107 described with reference to FIGS. 3A and 3B around a part with a need for rehabilitation. In this state, in accordance with a myoelectric potential generated in the subject attempting to move, a part with a need for assistance for rehabilitation is designated, and a voltage (electrical stimulus) as a supplement is applied to the part.

Firstly, the subject ties the bandage 107 around the part with a need for rehabilitation in the body. The stretchable bandage 107 is tied around the part and fixed with the attachment portion 108, such that the bandage 107 is fitted with a certain level of tightness. Subsequently, the subject attempts to move the part with a need for rehabilitation. In this practical example, it is assumed that a myoelectric potential, which can be a small amount, is generated in a rehabilitation target person when the person attempts to move a necessary part.

When the subject attempts to move the body, a myoelectric potential is generated, and as a result, the myoelectric potential is observed at a particular electrode 211 provided at the tied bandage 107 (step S201). The myoelectric potential is inputted to the input/output unit 205, and the input/output unit 205 switches to an electric potential input mode (step S202). The input/output unit 205 then transmits information about the myoelectric potential together with wiring line information (e.g., electrode identification information) to the identification attachment unit 206.

The identification attachment unit 206 detects the myoelectric potential information (step S203), designates an electrode at which the myoelectric potential is generated in accordance with the wiring line information, retrieves a corresponding electrode ID, and transmits the electrode ID together with the myoelectric potential information including information about current and voltage to the input unit 202. The input unit 202 transfers to the determination unit 203 the electrode ID and the myoelectric potential information transmitted from the identification attachment unit 206.

Next, the determination unit 203 designates a part in accordance with the electrode ID (step S204) and checks the myoelectric potential information of each electrode ID received from the input unit 202. When the voltage amplitude of the myoelectric potential does not exceed a predetermined threshold, the determination unit 203 determines to apply a voltage as a supplement (supplementary voltage) to the electrode corresponding to the electrode ID (step S205).

For the part designation according to the electrode ID, before the measurement, information about individual body parts and electrode IDs are inputted to the control device 200 and associated with each other. Alternatively, instead of designating a part, electrodes are fitted on the left and right sides of the body in the same manner and associated with each other, and an electrode to be fed with electricity is designated in accordance with information about differences between left and right electric potentials, as in a fourth practical example described later. The fourth practical example is easy to use especially because myoelectric potentials can be compared to each other by only fitting electrode cloths on the left and right sides in the same manner without previous consideration of which part electrodes should be fitted on.

As an example of how to determine whether a myoelectric potential exceeds the threshold, the following is a description of the case in which body parts and corresponding electrode IDs are associated with each other in advance. Firstly, a database of body parts associated with electrode IDs is created by, for example, visually checking which body parts IDs assigned to electrodes are fitted on. Additionally, a database (e.g., myoelectric potential database) of normal myoelectric potentials of individual body parts is previously created. This myoelectric potential database is created by associating body parts and normal myoelectric potentials. The myoelectric potential database may be created in accordance with average values of collected data of past patients and updated or may be created in accordance with average values of samples obtained from healthy people.

The predetermined threshold can be set in accordance with data of healthy people by using a known method. A voltage to be applied may be set by inputting a typical value usually used for supplementary voltage application for rehabilitation. The voltage to be applied may be set by inputting, for example, a value between a voltage necessary for healthy people and a voltage actually observed. Alternatively, the voltage (supplementary voltage) to be applied may be set to 80% of the voltage generated in healthy people. This is because applying voltage slightly lower than actually required voltage supports rehabilitation. Instead of setting the threshold, the observed myoelectric potential may be amplified at a fixed rate of amplification and applied as an electrical stimulus without variation.

Next, the input/output unit 205 switches from the voltage detection mode to the voltage application mode (step S207). Next, the signal generation unit 204 selects a wiring line associated with the electrode ID determined by the determination unit 203 and applies a necessary voltage determined by the determination unit 203 to the wiring line (step S208). The input/output unit 205 passes the voltage from the signal generation unit 204 to the designated electrode through the designated wiring line.

With this configuration, supplementary voltage (electrical stimulus) can be applied to the subject from an electrode close to the part the subject attempts to move. As such, the subject can make progress in recovery by being effectively supported in rehabilitation.

Additionally, the subject does not need to previously find a part of the body to be fed with electricity to fit an electrode on the part. Instead, for example, the subject fits a plurality of electrodes on the body by wearing the electrode cloth around the body; and in accordance with information about a myoelectric signal generated because the subject moves the body, electric power is applied from outside to supplement the myoelectric signal. Thus, the subject can easily do preparation.

With the configuration of this practical example, the cloth (electrode cloth) including a plurality of electrodes does not need to be prepared in a particular shape that fits the designation circuit of the body; the cloth in a normal shape capable of being fitted around any body part in any shape can cover almost all parts with a need for rehabilitation, which can reduce manufacturing costs and stock risks.

### Second Practical Example

Next, a second practical example will be described. The second practical example enables the subject to designate a part with a need for supplementary apply voltage by using, for example, a switch coupled to an electrode, and as a result, supplementary voltage can be applied to the particular part that the subject clearly desires to apply voltage to. For example, buttons (switches), which are not illustrated in the drawings, are individually coupled to the plurality of electrodes 211. The plurality of electrodes 211 have a function of, when a corresponding button is pressed, transmitting an application request information signal to the input/output unit 205 through a wiring line.

Any switch can be coupled to the electrode, but a switch that can be pressed, such as a push-button or pressure sensitive sensor, would be convenient because, when such a switch is provided on the electrode, the subject only needs to press a part that the subject desires to have support at so that supplementary voltage can be applied to the part.

Next, an operation of this practical example will be described. Firstly, the subject fits the cloth (electrode cloth) including the plurality of electrodes 211 around a part of the body with a need for rehabilitation. Next, the subject or a third person such as a medical doctor selects from the electrodes provided at the electrode cloth a button on an electrode at a part that the subject or third person desires to apply voltage to and presses the button. One or more buttons may be pressed.

The electrode coupled to the pressed button generates and transmits an application request information signal to the identification attachment unit 206 via the input/output unit 205. At this time, the input/output unit 205 detects the voltage from the electrode and switches to the detection mode. The identification attachment unit 206 obtains wiring line information from the input/output unit 205, retrieves a corresponding electrode ID, and transfers the electrode ID to the input unit 202.

The determination unit 203 determines to apply a given voltage to the electrode corresponding to the electrode ID via the input unit 202. The signal generation unit 204 applies voltage to the electrode indicated by an instruction received from the determination unit 203. To apply this voltage, the input/output unit 205 switches to the application mode. The voltage to be applied and the time for application are determined in accordance with information about the voltage and time received from the external interface (IF), which is not illustrated in the drawings. This means that the subject or medical doctors can freely set the level of voltage to be applied and the time for application by using the external IF.

As such, the given voltage can be applied from the signal generation unit 204 via the input/output unit 205 to the designated electrode, that is, the electrode coupled to the pressed button, which supports rehabilitation. Although the second practical example aims to support rehabilitation, this practical example can be used for health promotion purposes such as low-frequency therapy for, for example, healing stiff shoulders.

### Third Practical Example

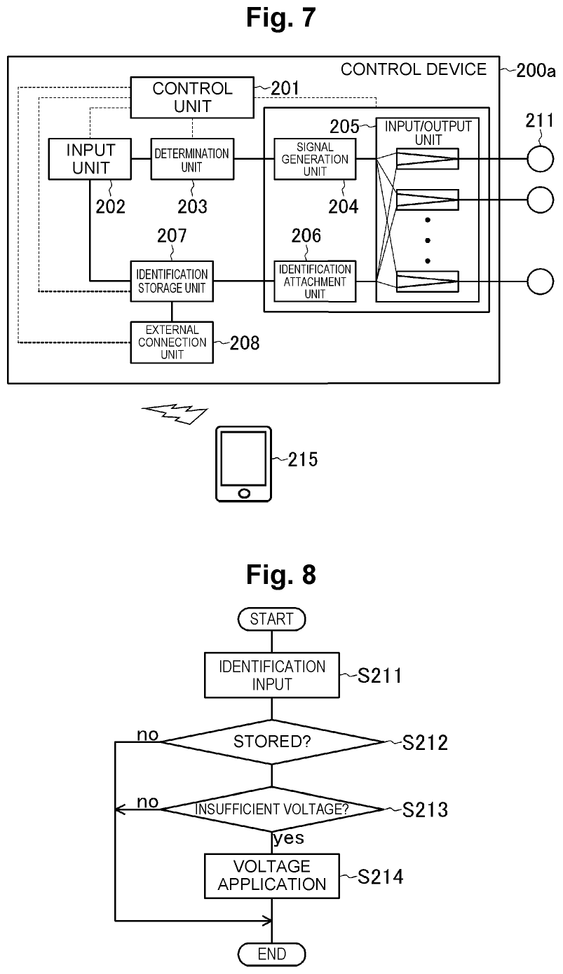

Next, a third practical example will be described with reference to FIG. 7. The third practical example is characterized in that: the subject wears the electrode cloth and previously designates an electrode to be fed with supplementary voltage; when the myoelectric potential at the electrode is insufficient, supplementary voltage is applied. In other words, an electrode is designated similarly to the second practical example, and voltage is applied when insufficient similarly to the first practical example. This configuration produces the effect in which no voltage is applied from electrodes at normal parts without any need for voltage application.

In a control device 200a according to the third practical example, the electrode ID determined by the identification attachment unit 206 is not received by the input unit 202 but received (stored) by an identification storage unit (storage circuit) 207. The identification storage unit 207 in advance stores the electrode ID of an electrode to be fed with electricity. The electrode ID to be stored in the identification storage unit 207 is received by using an external connection unit (external connection circuit) 208.

For example, two kinds of modes consisting of an ID storage mode and a measurement and application mode are provided at the external connection unit 208. When the subject selects the ID storage mode on the external connection unit 208 and presses a button of an electrode that the subject desires to apply voltage to, the electrode desired by the subject is designated and stored in the identification storage unit 207. When the measurement and application mode is selected, the identification storage unit 207 does not store the electrode ID of the electrode 211 coupled to a pressed button. The external connection unit 208 can be implemented by providing an interface at the control device 200a, and the subject can directly configure settings by using the interface. The external connection unit 208 may be configured to wirelessly communicate with an external terminal 215 by which settings can be configured. The external terminal 215 may be, for example, a mobile phone terminal or smartphone.

Next, an operation of the determination unit 203 according to the third practical example will be described with reference to FIG. 8. As described above, the identification storage unit 207 previously stores the electrode ID of an electrode that the subject desires to apply electricity to and that is designated by using the external connection unit 208.

When the subject moves the body for rehabilitation, a myoelectric potential is observed at one of the plurality of electrodes 211, the input/output unit 205 switches to the detection mode, and the observed myoelectric potential is transmitted with the electrode ID to the identification storage unit 207 via the identification attachment unit 206 (step S211).

The identification storage unit 207 determines whether the received electrode ID has been stored (step S212). When the received electrode ID has been stored, the identification storage unit 207 transmits the electrode ID together with the observed myoelectric potential information to the input unit 202.

The determination unit 203 receives the myoelectric potential information and the electrode ID via the input unit 202. When the voltage amplitude of the myoelectric potential does not exceed a predetermined threshold, the determination unit 203 determines to apply supplementary voltage to the electrode corresponding to the electrode ID (step S213). The flow after the determination for application to the application of voltage to the electrode 211 by using the signal generation unit 204 is identical to the flow in the first practical example (step S214).

This configuration enables the subject to designate a necessary part in the state in which the subject wears the electrode cloth around an affected part, and apply supplementary voltage to the necessary part without the possibility of applying voltage to unnecessary parts.

### Fourth Practical Example

Next, a fourth practical example will be described with reference to FIG. 7. The fourth practical example specifies an example of a method of determining whether the myoelectric potential at a part with a need for rehabilitation is insufficient. A known method of measuring myoelectric potential to determine whether support is needed measures myoelectric potential at the left and right side of the body (refer to FIG. 6 in Reference Literature 1). This technology is applied here.

The subject uses a plurality of electrode cloths or wears a wearable electrode cloth to measure myoelectric potential at both a normal part and a part with a need for rehabilitation. In the following description, the plurality of electrode cloths are coupled to the same control device 200. Because left and right myoelectric potentials are to be measured in this practical example, left and right corresponding electrodes need to be previously registered. The left and right myoelectric potentials denote a pair of myoelectric signals at positions symmetrical about the center line of a body. For example, in the case of arms, the left and right myoelectric potentials denote a myoelectric signal at a right arm and a myoelectric signal at a left arm. For this reason, the identification storage unit 207 previously stores left and right corresponding electrode IDs.

There are various conceivable methods for the registration. One method of designation using the external connection unit 208 is described here.

For example, two kinds of modes consisting of the ID storage mode and the measurement and application mode are provided at the external connection unit 208. When the subject selects the ID storage mode by using the external connection unit 208, the external connection unit 208 requests the subject to press buttons of two electrodes in a pair one by one.

The subject firstly presses a button of an electrode 211 of a right electrode cloth, and the identification storage unit 207 receives an electrode ID corresponding to the electrode 211 via the identification attachment unit 206. At this time, the external connection unit 208 requests the subject to press a subsequent button of a corresponding electrode 211 of a left electrode cloth. The subject presses a button of an electrode 211 of the left electrode cloth in response to this request, and the identification storage unit 207 receives an electrode ID via the identification attachment unit 206 and stores the electrode ID of the left electrode cloth in association with the previously received electrode ID of the right electrode cloth.

This configuration enables pairs of electrodes symmetrical about the center line of a body to be associated with each other in accordance with designation by the subject, regardless of how the subject fits the electrode cloth around the body. The subject successively selects (e.g., stores) electrode IDs of all surrounding electrodes that the subject desires to apply supplementary voltage (e.g., electrical stimulus) to. After all the necessary electrode IDs are associated with each other, the external connection unit 208 is switched from the ID storage mode to the measurement and application mode.

As such, the identification storage unit 207 previously stores electrode positions on the left and right sides of the subject in association with each other. The external connection unit 208 may have a wireless communication function. In this case, the association operation may be performed from outside by using, for example, the external terminal 215. Configuring settings by using the external terminal 215 enables high-speed processing with the use of a central processing unit (CPU) installed in the external terminal 215. Furthermore, because a user interface on a large screen of the external terminal 215 can be used, the designation operation is facilitated.

For example, the external terminal 215 displays a drawing of a human body on a screen and presents a request to press a button of an electrode of the right electrode cloth. When the subject presses a button of an electrode 211 of the right electrode cloth, the external terminal 215 submits a request to display a position of the electrode 211 of the pressed button on the human body displayed on the screen of the external terminal 215 by using the external connection unit 208. Subsequently, the external terminal 215 presents a request to press a button of a corresponding electrode of the left electrode cloth. When the subject presses a button of an electrode 211 of the left electrode cloth, the external terminal 215 submits a request to display a position of the electrode 211 of the pressed button on the human body displayed on the screen of the external terminal 215 by using the external connection unit 208. This configuration enables the subject to easily register a pair of two corresponding electrodes on the screen of the external terminal 215. The external terminal 215 stores information about an affected part together with the electrode ID in accordance with the operations of the subject described above. Sixth and seventh practical examples will describe how to use the part (affected part) of the subject.

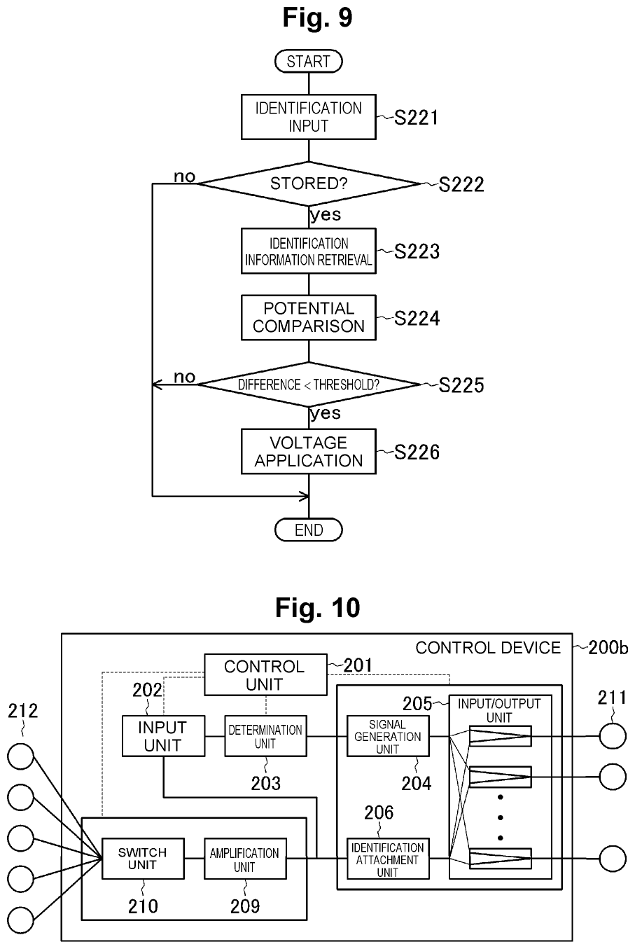

Next, an operation of the fourth practical example will be described with reference to FIG. 9. It is assumed that the subject has fitted electrode cloths on the left and right sides of the body and has associated left and right positions of electrodes that the subject desires to apply supplementary electrical stimuli to. After the subject finishes the preparation operation, the subject selects parts with a need for rehabilitation for either left or right part and attempts to move the left and right parts in the same manner on the assumption that either left or right part is in a normal condition. Specifically, when the selected parts are arms, for example, the subject moves up and down both arms at the same time.

Firstly, the identification attachment unit 206 attaches an ID of an electrode of the plurality of electrodes 211 at which the generation of myoelectric potential is observed to myoelectric potential information and transmits the ID and myoelectric potential information to the identification storage unit 207 (step S221). Next, the identification storage unit 207 checks whether the received electrode ID is stored in the identification storage unit 207 (step S222). When the received electrode ID is not stored in the identification storage unit 207, the operation ends at this point (no in step S222). By contrast, when the received electrode ID is stored in the identification storage unit 207 (yes in step S222), the identification storage unit 207 transmits the electrode ID together with myoelectric potential information to the input unit 202.

The determination unit 203 receives via the input unit 202 the electrode ID at which a myoelectric potential is generated. Next, the determination unit 203 retrieves an electrode ID constituting a pair with the received electrode ID from the identification storage unit 207 (step S223) and compares myoelectric potentials generated at the two electrode IDs (step S224). This means it is assumed that myoelectric potential is generated at a plurality of electrodes at this time, and myoelectric potential is observed in at least two electrodes associated with each other in the ID storage circuit.

As the result of this comparison, when the difference of measurement result between left and right myoelectric potentials is equal to or less than a given threshold, the process ends without performing a subsequent operation (no in step S225). By contrast, when the difference of measurement results between left and right myoelectric potentials exceeds the threshold (yes in step S225), an instruction is provided to apply voltage to an electrode corresponding to an electrode ID with a relatively low voltage (step S226). This is applied to the case in which myoelectric potential is generated at either of the electrode IDs stored in the identification storage unit 207. As measurement results of myoelectric potential, amplitude, pulse width, and phase are utilized (refer to FIG. 3 in Reference Literature 1).

The threshold can be set in any manner as appropriate. For example, in some cases the voltage (e.g., myoelectric potential) of one electrode of associated electrodes may be equal to or lower than half of the voltage of the other electrode of the associated electrodes. The voltage (e.g., electrical stimulus) to be applied can be any level of voltage. It is desirable to use a voltage usually used for rehabilitation. For example, the voltage to be applied can be set between the myoelectric potential at one electrode and the myoelectric potential at the other electrode; more specifically, the voltage to be applied can be set to 80% of the voltage generated in healthy people, that is, the higher voltage. This is because applying voltage slightly lower than actually required voltage as an electrical stimulus supports rehabilitation.

The fourth practical example measures myoelectric potentials in a symmetrical relationship about the center line of a body to designate a necessary application part. This eliminates a risk of variations in generation of myoelectric potential due to individual variations. Moreover, the fourth practical example uses an electrode cloth for measurement and application, and as a result, the same product can be used for most parts of human bodies.

### Fifth Practical Example

Next, a fifth practical example will be described with reference to FIG. 10. The fifth practical example causes the subject to sense myoelectric potential measured by using electrodes. The subject can thus recognize that myoelectric potential is generated even at a part that appears to be still or only move a little, and as a result, the subject can receive feedback. As such, the fifth practical example aims to facilitate rehabilitation. A control device 200b illustrated in FIG. 10 performs operations from measuring myoelectric potential at one of the plurality of electrodes 211 to attaching an electrode ID by the identification attachment unit 206 through the input/output unit 205 in the same manner as the practical examples described above.

The identification attachment unit 206 transmits the electrode ID and the measured myoelectric potential to an amplification unit (e.g., amplifier circuit) 209. The amplification unit 209 amplifies electric power in accordance with the voltage value of the received myoelectric potential to generate electric power of a level that enables a sign output unit (e.g., sign output devices) 212 to operate. A switch unit (e.g., switch circuit) 210 receives an electrode ID from the amplification unit 209 and connects a wiring line to a sign output unit 212 corresponding to the electrode ID. The sign output unit 212 may be constituted by, for example, a light-emitting diode. The sign output unit 212 may be constituted by a device for outputting sign information such as a speaker unit. It is preferable that the sign output units 212 be arranged at positions corresponding to the electrodes 211.

As an example of an operation of this practical example, firstly, the subject wears an electrode cloth and performs a rehabilitation activity. During rehabilitation, the subject cannot always move the body as the subject expects. However, the subject's intention of moving the body is outputted as a signal from the brain and may reach an affected part to a small extent. One of the plurality of electrodes 211 receives this signal as a myoelectric potential and transmits the myoelectric potential via the input/output unit 205 to the identification attachment unit 206. The identification attachment unit 206 attaches an electrode ID corresponding to the electrode and transmits the electrode ID together with the measured myoelectric potential information to the amplification unit 209. In accordance with the received myoelectric potential information, the amplification unit 209 generates electric power that can cause the sign output unit 212 constituted by a light-emitting diode to emit light. The amplification unit 209 then transmits the electric power together with the electrode ID to the switch unit 210. The switch unit 210 transfers the received electric power to a sign output unit 212 associated with the electrode ID.

In consideration of combination with the first practical example, the identification attachment unit 206 may transmit the electrode ID and the myoelectric potential information not only to the amplification unit 209 but also to the input unit 202, and electrical stimuli can be at the same time applied to the electrode 211 by using the determination unit 203.

This configuration enables the subject of rehabilitation to visually recognize that a signal is surely transmitted to a part that does not move although the subject attempts to move. It can be expected that such visual feedback further facilitates rehabilitation.

### Sixth Practical Example

Next, software configured to run on the external terminal 215 will be described. This is for motivating the subject to perform rehabilitation.

Rehabilitation subjects including many elderly people perform rehabilitation under instructions from instructors at rehabilitation facilities. But after the subjects switch to the stage of home care, a problem arises in which the subjects' motivation for rehabilitation decreases to prevent recovery. This practical example attempts to address the problem.

In the third and fourth practical examples, the user interface of the external terminal 215 is used to designate a part of an electrode to be fed with an electrical stimulus, that is, a part with a need for rehabilitation. The designation of electrode is performed in the ID storage mode of the external connection unit 208. However, in this practical example, a myoelectric potential corresponding to the stored electrode ID is transmitted via the external connection unit 208 to the external terminal 215 in the measurement and application mode.

The external terminal 215 receives and stores information about an actual myoelectric potential in association with information about a part with a need for rehabilitation. In the third practical example, the external terminal 215 stores information about a myoelectric potential at an affected part; in the fourth practical example, the external terminal 215 stores the information about the myoelectric potential at the affected part and information about a myoelectric potential at the other of the left and right parts corresponding to the affected part.

To reduce the amount of data, not all myoelectric potentials but sampling data or an average of values obtained for a given measurement period may be stored. This practical example uses such myoelectric potential information as an evaluation value.

These positional information of an affected part and myoelectric potential information are stored with a date and time of measurement. The software of this practical example implements means (step) of displaying these kinds of information in chronological order. Specifically, the software of this practical example implements means of averaging myoelectric potential information and displaying the averaged myoelectric potential information with a date and time of measurement in chronological order. This configuration can address an issue in which subjects cannot realize recovery for a medium to long term, and thus, it can be expected that daily increases in myoelectric potential encourage subjects to gain interests in rehabilitation.

### Seventh Practical Example

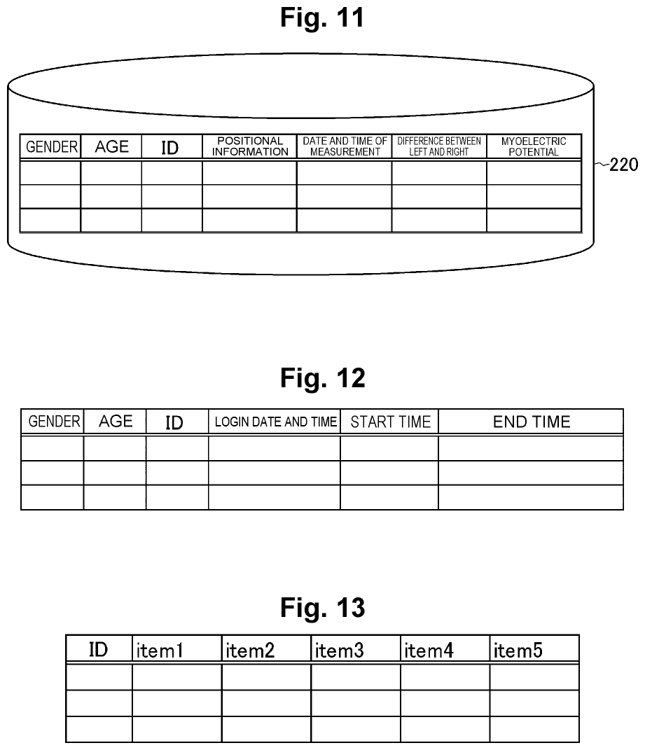

Next, the seventh practical example of the present invention will be described with reference to FIG. 11. The seventh practical example relates to a server 220 in a network that cooperates with software configured to run on the external terminal 215 and also relates to a method for increasing subject's motivation for rehabilitation.

Rehabilitation subjects including many elderly people are forced to perform rehabilitation at rehabilitation facilities. But after the subjects switch to the stage of home care, a problem arises in which the subjects' motivation for rehabilitation decreases to prevent recovery. Another problem is that many elderly people cannot gain interests in rehabilitation because they live alone and do not have communication with people in local communities. This practical example attempts to address the problem.

The sixth practical example exhibits time series changes in rehabilitation by displaying rehabilitation parts and the amounts of apply voltage on the external terminal 215, and as a result, chronological improvements and recovery can increase subject's motivation. The seventh practical example additionally shares these kinds of information with other people in expectation of increase in motivation.

The external terminal 215 transmits information about date and time of measurement, information about apply voltage, and information about application part, which are recorded during a rehabilitation activity, to the server 220 in a network by using software installed in the external terminal 215. The information may be transmitted in real time whenever the measurement is performed in rehabilitation or transmitted in a batch once a day. Alternatively, the information may be transmitted when the subject starts an application on the external terminal 215.

The server 220 stores the information about date and time of measurement, information about apply voltage, and information about application part in association with an ID of the subject. In addition to the ID, information including address, name, gender, and age may be stored together as personally identifiable information of the subject. The information about age and gender is helpful in searching for people in conditions similar to the condition of a particular person. The server 220 stores these kinds of chronological information in association with registered identification information of the subject.

Next, an operation of the server 220 according to this practical example will be described. The subject sends a request to search for a person in a condition similar to the condition of the subject from the external terminal 215 to the server 220. The person in a similar condition is, for example, a person having a similar part fed with voltage for rehabilitation.

The server 220 retrieves information about a particular part of the subject fed with voltage in accordance with the ID of the subject, extracts information about date and time of rehabilitation, information about application part, and information about apply voltage of another subject having a part identical or similar to the particular part, and creates a time series graph. The created graph is transmitted to the external terminal 215 and displayed on a display device of the external terminal 215. Similar parts denote parts close to each other, such as upper and lower arms or an entire lower limb and toes. These parts are previously registered as similar part information in the server 220 so that these parts can be searched for at any time by referring to data.

With the configuration described above, the subject can view the graph displayed on the display device of the external terminal 215 and recognize that the person in a condition similar to the condition of the subject performs rehabilitation on a daily basis without skipping rehabilitation and appears to be recovering, which may raise subject's hope for the future.

As the information to be extracted, information about age and gender may be included in search targets. Because the degree of recovery varies in accordance with age and gender, the subject can refer to data of other people with affinity. These kinds of search information can be search keys only when the subject desires to search such information and other subjects providing data agree with the search. One conceivable method for obtaining approval is, for example, sending a request for approval for data sharing as search results from the server 220 to other subjects providing data. At this time, the request for approval sent from the server 220 may include information such as age, gender, and affected part, which do not identify a particular subject requested to share data but infer the condition of the particular subject.

Such information acts as a factor in decision making more importantly than personally identifiable information, because shared data of such information about people in similar conditions may provide elderly people with a chance of widening their lives without limiting their lives in rehabilitation, although elderly people in particular are likely to have less motivation for rehabilitation because they feel lonely and isolated.

When a subject sends a request for data sharing to another subject and the other subject accepts the request, the server 220 associates an ID of the subject sending the request for data sharing with an ID of the other subject requested to share data and stores the IDs with a share flag. The share flag is used to determine whether to include information of one of the subjects when search results are displayed in response to requests for search from the other of the subjects.

An additional function may be provided in which a subject viewing similar condition data can send a message through the server 220 to another rehabilitation subject in a similar condition. It can be expected that the mutual encouragement of people in the same condition can increase motivation for rehabilitation and also produce chances of forming new communities.

### Eighth Embodiment

Next, an eighth practical example of the present invention will be described. This practical example is formed by adding a gaming function for increasing motivation to continue rehabilitation to the sixth and seventh practical examples. The server 220 described with reference to FIG. 11 stores, in addition to information according to the data fields indicated in FIG. 11, information about gender and age, and also information about login date and time of subject, rehabilitation start time, and rehabilitation end time in association with ID, as indicated in FIG. 12. An earliest time of date and time of measurement of rehabilitation is stored as the rehabilitation start time. A latest time of date and time of measurement of rehabilitation is stored as the rehabilitation end time.

FIG. 13 illustrates a table for associating information of subjects' IDs with information of IDs (e.g., item ID) of various items. The various items here denote virtual items presented as rewards for subjects on software. The various items can be any items including medals, puzzle pieces, and accessories for avatars. Particularly, when the various items are related to each other and a collection of some items represents a collective meaning, subjects' motivation for collection increases, and they are also motivated to exchange items with others.

One conceivable example of relating the various items to each other is that different colors of medals are ranked, for example, as follows: three copper medals changes to silver; or three silver medals changes to gold. Another conceivable example is that the items for avatars are all accessories worn by characters, such as sunglasses, hats, jackets, and skirts. Still another conceivable example is that the items relate to each other to form one object; for example, the items are puzzle pieces having different roles and the puzzle pieces together form one drawing.

The following describes how items are expressed when subjects possess the items, with reference to FIG. 13. For example, in the case in which the item ID of gold medal is 1, when 1 is assigned as a flag to item 1, one gold medal is possessed; when o is assigned as a flag to item 1, no gold medal is possessed. Next, an operation for possessing various items will be described. To increase motivation for collection of various items for the purpose of promoting participation in rehabilitation, opportunities to obtain the items can be determined in accordance with subject's status in rehabilitation activities.

One method for determining whether the subject can obtain items is according to, for example, the number of consecutive login days. This is because the continuity of rehabilitation is important. Rehabilitation is more effective when it is continuously performed than when it is performed with interruptions. For example, an opportunity to obtain one item is given for three consecutive login days.

However, actual rehabilitation activities cannot be proved by only the login information. Thus, when the period between a rehabilitation start time and a rehabilitation end time indicated as an example in FIG. 12 is a given period, such as thirty minutes or more, one rehabilitation activity is counted; when a predetermined number of rehabilitation activities have been performed, an opportunity to obtain items is given. This is because performing rehabilitation in a continuous manner and for a certain time period is important.

The server 220 has a function of determining such item acquisition opportunities. When the server 220 determines that a subject satisfies a condition for an item acquisition opportunity, the server 220 provides an item for the ID of the subject. Specifically, 1 is assigned to one item ID. A particular item to be obtained (provided) is determined in accordance with random numbers. The items may differ from each other to some extent in terms of how easy the items can be obtained. Some items may be easily obtained, whereas other items may be rarely obtained. As described above, the items are provided in accordance with rehabilitation status of individual subjects.

The server 220 extracts flag statuses of item IDs described above with respect to each subject ID and transmits a display instruction to the external terminal 215 of the subject. The external terminal 215 reads images corresponding to the item IDs from a storage circuit of the external terminal 215 or the server 220 and displays the images on the screen of the external terminal 215. For example, when the various items are puzzle pieces, images of the items owned by the subject are displayed at particular positions in one drawing. The subject can view on the screen the own various items collected by the subject, and as a result, motivation for collection is increased to gain more motivation for rehabilitation.

As described above, subjects can acquire various items in accordance with rehabilitation status. By combining the seventh and eighth practical examples together, further increase in motivation for rehabilitation can be expected. The seventh practical example describes formation of community with other people who make efforts to perform rehabilitation in a similar manner. The eighth practical example describes improvement of motivation by collecting various items. In the example described above, items to be provided are determined in accordance with random numbers regardless of subjects' interest. As a result, subjects may acquire unwanted items or multiple identical items.

However, other people may desire to have these items, and thus, items may be exchanged in the communities. Specifically, at least two subjects mutually confirm intentions by using a communication function and select items for exchange. Any communication function can be used. For example, the function of sending messages described in the seventh practical example may be used.

The selected items are exchanged by changing numerals in the fields of corresponding item IDs associated with the IDs of the subjects in the server 220. For example, item 1 of subject A is increased by 1, and item 1 of subject B is decreased by 1; item 2 of subject A is decreased by 1, and item 2 of subject B is increased by 1; accordingly, item 2 of subject A is exchanged for item 1 of subject B.

The eight practical examples have been described above. These practical examples can be changed as appropriate without changing the spirit of the present invention. Furthermore, no limitation exists when these practical examples are combined together for application.

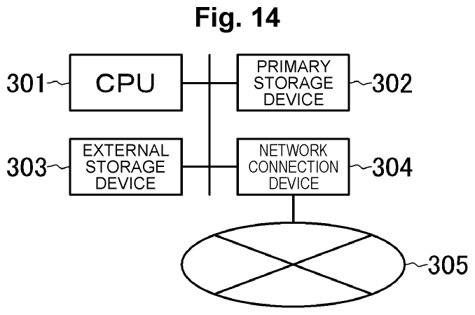

The designation circuit, the application circuit, the storage circuit, and the receive circuit, which constitute the electrical stimulation device according to the embodiment, may be implemented as a computer device including, for example, a central processing unit (CPU) 301, a primary storage device 302, an external storage device 303, and a network connection device 304 as illustrated in FIG. 14. The functions (e.g., electrical stimulation application) described above may be implemented by the CPU 301 operating (e.g., running a program) in accordance with a program loaded on the primary storage device 302. The program is used by the computer to implement the electrical stimulation method described in the embodiment. The network connection device 304 is connected to a network 305. The functions may be separately provided in a plurality of computer devices.

The electrical stimulation device according to the embodiment may be implemented as a programmable logic device (PLD) such as a field-programmable gate array (FPGA). For example, the designation circuit, the application circuit, the storage circuit, and the receive circuit are provided in an FPGA logic element to implement the electrical stimulation device. The designation circuit, the application circuit, the storage circuit, and the receive circuit can be configured on the FPGA by using a given configuration device connected. The circuits configured on the FPGA can be checked by using the configuration device connected to the FPGA.

As described above, in embodiments of the present invention, an electrode at which myoelectric potential is measured is designated from the plurality of electrodes for measuring myoelectric potential in the state in which the plurality of electrodes are fitted on the surface of the body of a target person, and a signal is applied to the designated electrode. As a result, it is possible to relatively easily apply electrical stimuli to a part with a need for rehabilitation.

It should be noted that the present invention is not limited to the embodiments described above, and it is apparent that many kinds of modifications and combinations can be made by those skilled in the art without departing from the spirit and scope of the present invention.

Reference Literature 1: Clinical application of electromyogram, (2) Evaluation of muscle recruitment with surface electromyogram, [searched on Jul. 5, 2019], (https://www.sakaimed.co.jp/knowlege/surface-electromyogram/clinical/clinical02/).

### REFERENCE SIGNS LIST

100 Electrical stimulation application device
101 Electrodes
102 Designation circuit
103 Application circuit
104 Storage circuit
105 Receive circuit

1.-8. (canceled)

9. An electrical stimulation device comprising:
a plurality of electrodes for measuring myoelectric potential in a state in which the plurality of electrodes are fitted on a body surface of a target person;
a designation circuit configured to designate, from the plurality of electrodes, an electrode at which myoelectric potential is measured; and
an application circuit configured to apply a signal to the electrode designated by the designation circuit.

10. The electrical stimulation device of claim 9 wherein the designation circuit is configured to designate, from the plurality of electrodes, the electrode when the myoelectric potential is higher than a preset reference value.

11. The electrical stimulation device of claim 9 further comprising:
a storage circuit configured to store identification information for identifying an electrode targeted for application,
wherein the application circuit is configured to apply a signal to an electrode that is identified by the identification information stored in the storage circuit and that is designated by the designation circuit.

12. The electrical stimulation device of claim 11 further comprising:
a receive circuit configured to receive an instruction about a target electrode of the plurality of electrodes, and to store in the storage circuit the identification information identifying the target electrode of the received instruction.

13. An electrical stimulation application method comprising:
designating an electrode at which myoelectric potential is measured from a plurality of electrodes for measuring myoelectric potential in a state in which the plurality of electrodes are fitted on a body surface of a target person; and
applying a signal to the electrode that is designated.

14. The electrical stimulation application method of claim 13, wherein designating the electrode comprises:
designating the electrode when the myoelectric potential is higher than a preset reference value.

15. The electrical stimulation application method of claim 13, wherein applying the signal comprises:
applying a signal to an electrode that is identified by identification information stored in a storage circuit and that is designated.

16. The electrical stimulation application method of claim 15 further comprising:
receiving an instruction about a target electrode of the plurality of electrodes; and
storing in the storage circuit the identification information identifying the target electrode of the received instruction.

17. An electrical stimulation device comprising:
a cloth;
a plurality of electrodes on the cloth;
a control circuit configured to:
designate a first electrode of the electrodes at which myoelectric potential is measured when the electrodes are fitted on a body surface of a target person;
determine a body part of the target person associated with the first electrode;
determine a normal myoelectric potential for the body part of the target person; and
apply a signal to the first electrode when the measured myoelectric potential does not exceed the normal myoelectric potential.

18. The electrical stimulation device of claim 17, wherein the cloth is a stretchable bandage.
